(19) United States
(12) Patent Application Publication
(10) Pub. No.: US 2023/0190993 A1
(43) Pub. Date: Jun. 22, 2023

(54) FIBRINOGEN COMPRISING FORMULATION AND USES THEREOF

(71) Applicants: Omrix Biopharmaceuticals Ltd., Kiryat-Ono (IL); Ethicon, Inc., Sommerville, NJ (US)

(72) Inventors: NINA RAVER SHAPIRA, REHOVOT (IL); ASHLEY DEANGLIS, SKILLMAN, NJ (US); OMRI FAINGOLD, REHOVOT (IL)

(21) Appl. No.: 18/080,815

(22) Filed: Dec. 14, 2022

Related U.S. Application Data
(60) Provisional application No. 63/292,173, filed on Dec. 21, 2021.

(51) Int. Cl. A61L 24/04; A61L 15/22; A61L 15/42

(52) U.S. Cl. CPC A61L 24/043; A61L 15/225; A61L 15/425; A61L 2400/04

(57) ABSTRACT

The invention relates to compositions comprised of albumin and clottable proteins including fibrinogen and to use thereof e.g., for treating bleeding. In particular, the compositions are comprised of albumin and one or more clottable proteins, wherein the albumin and the one or more clottable proteins are present at a total concentration of at least 90% by total protein weight, wherein the clottable protein fibronectin is present at an amount of less than about 0.5% by total protein weight or is absent, and wherein the weight ratio of the albumin to the clottable protein fibrinogen is at least 1:15, respectively.

### TECHNOLOGICAL FIELD

The present disclosure relates to compositions comprising fibrinogen. The present invention relates, inter alia, to fibrinogen preparations which include albumin and very low amounts of fibronectin.

### BACKGROUND ART

References considered to be relevant as background to the presently disclosed subject matter are listed below:
U.S. Pat. No. 7,241,603
US Patent Application No. 2016/0015792
U.S. Pat. No. 9,084,728
U.S. Pat. No. 8,962,025
U.S. Pat. No. 10,111,980
U.S. Pat. No. 9,056,092
International Application Publication No. WO2000/009018
International Application Publication No. WO1992/013495
US Patent Application No. 2020/0054720
Bar, L., et al. The binding of fibrin sealant to collagen is influenced by the method of purification and the cross-linked fibrinogen-fibronectin (heteronectin) content of the ‘fibrinogen’ component, Blood Coagulation & Fibrinolysis: 2005, 16(2), 111-117.

Acknowledgement of the above references herein is not to be inferred as meaning that these are in any way relevant to the patentability of the presently disclosed subject matter.

### BACKGROUND

Control of bleeding is essential and critical in surgical procedures to minimize blood loss. Biological glue/sealants being used as tissue adhesive or for control of hemostasis are described in the art.

Fibrinogen plays an important part in the clotting cascade and is considered as a key component in a variety of hemostatic and sealing products including, inter alia, fibrin patch and fibrin sealant. Current fibrinogen products include in addition to fibrinogen, other components that are purified from plasma together with fibrinogen. These compounds include for example coagulation factors such as factor VIII, factor XIII, von Willebrand factor (vWF) as well as fibronectin, the latter was previously shown to be essential in promoting tissue adhesion (Bar, L., et al., Blood Coagulation & Fibrinolysis: 2005, 16(2), 111-117).

U.S. Pat. No. 7,241,603 describes a method for producing protein compositions comprising fibrinogen and fibronectin, wherein a fibrinogen and fibronectin-containing starting solution is treated with a precipitating composition so that in a single-step precipitation a precipitate is formed which comprises fibrinogen and fibronectin, and the precipitate formed optionally is further treated by methods known per se.

US Patent Application No. 2016/0015792 relates to sterile powder compositions suitable for medical use comprising thrombin and fibrinogen, and to methods for producing the same.

U.S. Pat. No. 9,084,728 describes a process for making a dry and stable hemostatic composition.

U.S. Pat. No. 8,962,025 describes compositions, methods, and kits for sealing applications.

U.S. Pat. No. 10,111,980 describes a dry composition comprising one or more polyols, which upon addition of an aqueous medium forms a substantially homogenous paste suitable for use in haemostasis procedures.

U.S. Pat. No. 9,056,092 describes a hemostatic pad comprising a bioabsorbable scaffolding material; a lyophilized thrombin powder, a lyophilized fibrinogen powder, and a meltable binder powder, with all powders disposed on the bioabsorbable scaffolding material.

International Application Publication No. WO2000/009018 describes polymerized type I and/or III collagen-based compositions for medical use as vascular sealants and wound dressings, and the preparation thereof.

International Application Publication No. WO1992/013495 describes a fibrinogen based composition which is derived from bovine plasma through the use of polyethylene glycol (“PEG”) and glycine precipitation techniques.

US Patent Application No. 2020/0054720 provides methods for large scale preparation of sterile stable liquid thrombin composition comprised of glycerol; stable liquid thrombin compositions; and hemostatic composition and kits.

Bar, L. et al. (2005) describes fibrinogen preparations and shows by use of experiments with highly purified proteins that fibronectin was essential in promoting progressive binding of fibrinogen to collagen under the action of activated factor XIII (transglutaminase).

### GENERAL DESCRIPTION

In accordance with some aspects, the present disclosure provides a composition comprising one or more clottable proteins and albumin, wherein the one or more clottable proteins and the albumin are present at a total concentration of at least 90% by total protein weight, wherein the one or more clottable proteins comprise fibrinogen and fibronectin, with the fibronectin being at a concentration of less than about 0.5% by total protein weight or being absent and wherein the albumin to the fibrinogen weight ratio is at least 1:15, respectively.

In accordance with some aspects, the present disclosure provides a composition comprising one or more clottable proteins, albumin and thrombin, wherein the one or more clottable proteins and the albumin are present at a total concentration of at least 90% by total protein weight, wherein the one or more clottable proteins comprise fibrinogen and fibronectin, with the fibronectin being at a concentration of less than about 0.5% by total protein weight or being absent and wherein the albumin to the fibrinogen weight ratio is at least 1:15, respectively.

In accordance with some aspects, the present disclosure provides an article of manufacture comprising a composition having one or more clottable proteins and albumin, wherein the one or more clottable proteins and the albumin are present at a total concentration of at least 90% by total protein weight, wherein the one or more clottable proteins comprise fibrinogen and fibronectin, with the fibronectin being at a concentration of less than about 0.5% by total protein weight or being absent, and wherein the albumin to the fibrinogen weight ratio is at least 1:15, respectively, and wherein the composition is in a powder form.

In accordance with some embodiments, the article of manufacture is a wound dressing.

In accordance with some further aspects, the present disclosure provides a composition comprising one or more clottable proteins and albumin, wherein the one or more clottable proteins and the albumin are present at a total concentration of at least 90% by total protein weight, wherein the one or more clottable proteins comprise fibrinogen and fibronectin, with the fibronectin being at a concentration of less than about 0.5% by total protein weight or being absent, and wherein the albumin to the fibrinogen weight ratio is at least 1:15, respectively. The present disclosure further provides an article comprising the composition, e.g., for use in tissue adhesive.

In accordance with some further aspects, the present disclosure provides a composition comprising one or more clottable proteins and albumin, wherein the one or more clottable proteins and the albumin are present at a total concentration of at least 90% by total protein weight, wherein the one or more clottable proteins comprise fibrinogen and fibronectin, with the fibronectin being at a concentration of less than about 0.5% by total protein weight or being absent, and wherein the albumin to the fibrinogen weight ratio is at least 1:15, respectively. The present disclosure further provides an article of comprising the composition, for use in treatment, inhibition, arresting or delaying of bleeding or any disorder associated thereto.

In accordance with some other aspects, the present disclosure provides a method for the treatment, inhibition, arresting or delaying bleeding or any disorder associated thereto, the method comprising topically administering onto a wounded tissue of a subject in need thereof an effective amount of the composition comprising one or more clottable proteins and albumin, wherein the one or more clottable proteins and the albumin are present at a total concentration of at least 90% by total protein weight, wherein the one or more clottable proteins comprise fibrinogen and fibronectin, with the fibronectin being at an amount of less than about 0.5% by total protein weight or being absent, and wherein the albumin to the fibrinogen weight ratio is at least 1:15, respectively. The present disclosure further provides an article of comprising the composition, to thereby treat, inhibit, arrest or delay bleeding or any disorder associated thereto.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a graph showing tissue adherence of exemplary samples as a function of % albumin to total protein using a tissue peel force test, the error bars represent standard deviation, p values were calculated by a pooled t-test.

FIG. 2 is a bar graph showing tissue peel force as a function of % fibrinogen to total protein using a tissue peel force test, the error bars represent standard deviation.

FIG. 3 is a bar graph showing tissue peel force as a function of % albumin, the Y axis shows mean tissue peel force (N/meters) and the X axis the % of albumin, the error bars represent standard deviation.

FIG. 4 is an image showing an exemplary wound dressing comprising a fibrinogen composition pressed on resected Kidney

### DETAILED DESCRIPTION OF EMBODIMENTS

Control of bleeding is critical, for example, in surgical procedures to minimize blood loss, reduce post-surgical complications, and to shorten duration of surgery in the operation room.

The present inventors surprisingly found that pure fibrinogen preparations which include high amounts of fibrinogen and very low amounts of fibronectin and at times even essentially free of fibronectin, are highly effective and achieve strong tissue adherence as well as initiate hemostasis activity when combined with albumin. This low amount of fibronectin (if present at all), among other characteristics, distinguishes the fibrinogen preparation of the present disclosure from known fibrinogen preparations.

Hence, the present disclosure relates to compositions comprising fibrinogen, albumin and low amounts of fibronectin and at times being essentially free of fibronectin. Such compositions may be referred to, at times, as pure fibrinogen compositions.

In accordance with some aspects, the present disclosure provides a composition comprising one or more clottable proteins and albumin, wherein the one or more clottable proteins and albumin are at a total concentration of at least 90% (w/w) by total protein weight, wherein the one or more clottable proteins comprise fibrinogen and fibronectin, wherein the fibronectin is at an amount of less than about 0.5% (w/w) by total protein weight or being absent, and wherein the albumin to fibrinogen weight ratio is at least 1:15, respectively.

This composition is at times denoted herein as “fibrinogen-comprising composition”.

In the following text, when referring to the compositions it is to be understood as also referring to the article of manufacture, methods and kits disclosed herein. Thus, whenever providing a feature with reference to the compositions, it is to be understood as defining the same feature with respect to the article of manufacture, methods and kits, mutatis mutandis.

As shown in Example 1 below, pure fibrinogen preparations which comprise low amounts of fibronectin achieved strong tissue adherence upon addition of albumin. As shown in Example 2, full hemostasis was achieved after administration of patches comprising the compositions described herein.

It was thus suggested that pure fibrinogen preparations that comprise albumin and low amounts of fibronectin or even at times being free of fibronectin are highly effective as they adhere well to tissue and achieve homeostasis.

As described herein, the composition comprises clottable proteins. In the context of the present disclosure a “clottable protein” encompasses one or more of the plasma proteins that is part of a formed blood clot.

Typically, the term “clottable protein” should be understood as not encompassing all enzymes that are activated as part of clot formation. Non-limiting examples of a clottable protein include fibrinogen, fibronectin, alpha 2 antiplasmin and vitronectin.

The content of clottable proteins in the composition can be calculated by any method known in the art. For example, the content of clottable proteins can be calculated from clottable and total protein determinations. The “clottable protein” can be determined by clotting a diluted sample with thrombin in the presence of calcium ions. The clot is washed, and the remaining protein content is measured. The measurement may be performed gravimetrically or by dissolving the clot in alkaline urea or other reagents, followed by a spectrophotometric protein assay. The “total protein” can be determined by a variety of analytical techniques including ultraviolet spectrophotometry, dye binding assays, e.g., Coomassie or Bradford, copper-based assays, e.g., biuret, Lowry and the bicinchoninic acid (BCA) assay, and immunological methods, among others.

The composition comprises also albumin. The content of albumin can be calculated by any method known in the art. For example, the content of albumin can be calculated by dye binding methods, e.g., bromocresol green and bromocresol purple, electrophoretic methods, and immunological methods, e.g., immunonephelometry.

In some embodiments, the composition comprises one or more clottable proteins and albumin at a total concentration of at least about 91% out of the total protein weight dry matter, at times of at least about 92%, at times of at least about 93%, at times of at least about 94%, at times of at least about 95%, at times of at least about 96%, at times of at least about 97%, at times of at least about 97.5%, at times of at least about 98%, at times of at least about 98.5%, at times of at least about 99%, at times of at least about 99.2%, at times of at least about 99.4%, at times of at least about 99.6% and at times of at least about 99.8% out of the total protein weight dry matter. In some embodiments, one or more clottable proteins and albumin are at a total concentration of at least about 95% out of the total protein weight dry matter.

In some embodiments, the fibrinogen-comprising composition comprises albumin and one or more clottable proteins at a total concentration of between about 90% and about 100% out of the total protein weight dry matter. In some embodiments, the fibrinogen-comprising composition comprises albumin and one or more clottable proteins at a total concentration of between about 90% and about 99.5% out of the total protein weight dry matter.

In some other embodiments, the fibrinogen-comprising composition comprises one or more clottable proteins and albumin at a total concentration of between about 90% and about 99.9% out of the total protein weight dry matter, at times between about 92% and about 99.9%, about 94% and about 99.9%, about 95% and about 99.9%, about 97% and about 99.9%, about 98% and about 99.9%, about 99% and about 99.9%, about 99.2% and about 99.9%, about 99.4% and about 99.9%, about 99.6% and about 99.9% out of the total protein weight dry matter.

In some embodiments, the fibrinogen-comprising composition comprises one or more clottable proteins and albumin at a total concentration of about 90% out of the total protein weight dry matter, at times of about 92%, at times of about 93%, at times of about 94%, at times of about 95%, at times of about 96%, at times of about 97%, at times of about 97.5%, at times of about 98%, at times of about 98.5%, at times of about 99%, at times of about 99.2%, at times of about 99.4%, at times of about 99.5%, at times of about 99.6%, at times of about 99.7%, at times of about 99.8% and at times of about 99.9% out of the total protein weight dry matter.

In some embodiments, the fibrinogen-comprising composition comprises one or more clottable proteins and albumin at a total concentration of 99.5% out of the total protein weight dry matter, at times of about 99.6%, at times of about 99.7%, at times of about 99.8%, at times of about 99.9% out of the total protein weight dry matter.

In some embodiments, the one or more clottable proteins comprise at least one of fibrinogen, fibronectin, alpha 2 antiplasmin, vitronectin or any combination thereof.

In some embodiments, the one or more clottable proteins comprise fibrinogen, fibronectin or a combination thereof.

In some embodiments, the one or more clottable proteins comprise or is fibrinogen.

In some embodiments, the fibrinogen-comprising composition comprises fibrinogen and albumin at a total concentration of at least about 91% out of the total protein weight dry matter, at times of at least about 92%, at times of at least about 93%, at times of at least about 94%, at times of at least about 95%, at times of at least about 96%, at times of at least about 97%, at times of at least about 97.5%, at times of at least about 98%, at times of at least about 98.5%, at times of at least about 99%, at times of at least about 99.2%, at times of at least about 99.4%, at times of at least about 99.6% and at times of at least about 99.8% out of the total protein weight dry matter. In some embodiments, fibrinogen and albumin are at a total concentration of at least about 95% out of the total protein weight dry matter.

In some embodiments, the fibrinogen-comprising composition comprises fibrinogen and albumin at a total concentration of between about 90% and about 100% out of the total protein weight dry matter. In some embodiments, the fibrinogen-comprising composition comprises fibrinogen and albumin at a total concentration of between about 90% and about 99.5% out of the total protein weight dry matter.

In some other embodiments, the fibrinogen-comprising composition comprises fibrinogen and albumin at a total concentration of between about 90% and about 99.9% out of the total protein weight dry matter, at times between about 92% and about 99.9%, about 94% and about 99.9%, about 95% and about 99.9%, about 97% and about 99.9%, about 98% and about 99.9%, about 99% and about 99.9%, about 99.2% and about 99.9%, about 99.4% and about 99.9%, about 99.6% and about 99.9% out of the total protein weight dry matter.

In some embodiments, the fibrinogen-comprising composition comprises fibrinogen and albumin at a total concentration of about 90% out of the total protein weight dry matter, at times of about 92%, at times of about 93%, at times of about 94%, at times of about 95%, at times of about 96%, at times of about 97%, at times of about 97.5%, at times of about 98%, at times of about 98.5%, at times of about 99%, at times of about 99.2%, at times of about 99.4%, at times of about 99.5%, at times of about 99.6%, at times of about 99.7%, at times of about 99.8% and at times of about 99.9% out of the total protein weight dry matter.

In some embodiments, the fibrinogen-comprising composition comprises fibrinogen and albumin at a total concentration of about 99.5% out of the total protein weight dry matter, at times of about 99.6%, at times of about 99.7%, at times of about 99.8%, at times of about 99.9%.

As described herein, the albumin to fibrinogen weight ratio in the fibrinogen-comprising composition is at least about 1 to 15 (0.067), respectively.

In some embodiments that may be considered as aspects of the present disclosure, there is provided a fibrinogen-comprising composition comprising one or more clottable proteins and albumin, wherein the one or more clottable proteins comprise fibrinogen and wherein the albumin to fibrinogen weight ratio is at least about 1:15, respectively.

In some embodiments, the fibrinogen-comprising composition comprises fibrinogen and albumin, with the respective albumin-to-fibrinogen weight ratio being at least about 1:14 (0.07), at times at least about 1:13 (0.076), at times at least about 1:12 (0.083), at times at least about 1:11 (0.09), at times at least about 1:10 (0.1), at times at least about 1:9 (0.11), at times at least about 1:8 (0.125), at times at least about 1:7 (0.14), at times at least about 1:6 (0.167), at times at least about 1:5 (0.2), at times at least about 1:4 (0.25), at times at least about 1:3.5 (0.28), at times at least about 1:3 (0.31), at times at least about 1:2.5 (0.4), at times at least 1:2 (0.5), at times about 1:1.6 (0.625), at times about 1:1.5 (0.66), at times at least about 1:1.4 (0.71), at times at least about 1:1.3 (0.77), and at times at least about 1:1.2 (0.83).

In some embodiments, the fibrinogen-comprising composition comprises fibrinogen and albumin and the respective albumin to fibrinogen weight ratio is between about 1:0.8 (1.25) and about 1:15 (0.067).

In some embodiments, the fibrinogen-comprising composition comprises fibrinogen and albumin and the albumin to fibrinogen weight ratio is between about 1:0.9 and about 1:15 (0.067), at times between about 1:1 and about 1:15, at times between about 1:1 and about 1:14, at times between about 1:1 and about 1:13, at times between about 1:1 and about 1:12, at times between about 1:1 and about 1:11, at times between about 1:1 and about 1:10, at times between about 1:1 and about 1:9, at times between about 1:1 and about 1:8, at times between about 1:1 and about 1:7, and at times between about 1:2 and about 1:7.

In some embodiments, the fibrinogen-comprising composition comprises fibrinogen and albumin and the albumin to fibrinogen weight ratio is about 1:1 and about 1:10, at times between about 1:1.6 and about 1:3.

In some embodiments, the fibrinogen-comprising composition comprises fibrinogen and albumin and the albumin to fibrinogen weight ratio is about 1:0.8, at times about 1:0.9, at times about 1:1, at times about 1:1.1, at times about 1:1.2, at times about 1:1.3, at times about 1:1.4, at times about 1:1.5, at times about 1:1.6, at times about 1:1.7, at times about 1.1.8, at times about 1:1.9, at times about 1:2, at times about 1:3, at times about 1:4, at times about 1:5, at times about 1:6, at times about 1:7, at times about 1:8, at times about 1:9, at times about 1:10, at times about 1:11, at times about 1:12, at times about 1:13, at times about 1:14, and at times about 1:15.

As described herein, addition of albumin enhanced adhesion. In some embodiments, the fibrinogen-comprising composition comprises between about 0.1% and about 55% albumin out of the total protein weight dry matter.

In some embodiments, the fibrinogen-comprising composition comprises between about 0.5% and about 55% albumin out of the total protein weight dry matter, at times between about 0.5% and about 53%, at times between about 0.5% and about 50%, at times between about 0.5% and about 48%, at times between about 0.5% and about 45%, at times between about 0.5% and about 43% albumin out of the total weight of proteins in the composition on dry basis.

In some embodiments, the fibrinogen-comprising composition comprises between about 1% and about 55% out of the total protein weight dry matter, at times between about 3% and about 55%, at times between about 4.5% and about 55%, at times between about 5% and about 53%, at times between about 5% and about 50%, at times between about 5% and about 48%, at times between about 5% and about 45%, at times between about 5% and about 43% albumin out of the total amount of proteins in the composition on dry basis.

In some embodiments, the fibrinogen-comprising composition comprises between about 10% and about 55% out of the total protein weight dry matter, at times between about 10% and about 53%, at times between about 10% and about 50%, at times between about 10% and about 48%, at times between about 10% and about 45%, at times between about 10% and about 43% albumin out of the total amount of proteins in the composition on dry basis.

In some embodiments, the fibrinogen-comprising composition comprises between about 10% and about 55% out of the total protein weight dry matter, at times between about 12% and about 55%, at times between about 15% and about 55%, at times between about 18% and about 55%, at times between about 20% and about 55% albumin out of the total amount of proteins in the composition on dry basis.

In some embodiments, the fibrinogen-comprising composition comprises between about 10% and about 55% out of the total protein weight dry matter, at times between about 12% and about 53%, at times between about 14% and about 50%, at times between about 16% and about 48%, at times between about 18% and about 45%, at times between about 20% and about 40%, at times between about 23% and about 40% albumin out of the total amount of proteins in the composition on dry basis.

In some embodiments, the fibrinogen-comprising composition comprises about 1% (1 mg/ml) albumin, at times about 5% about, at times about 10%, at times about 12%, at times about 15%, at times about 17%, at times about 20%, at times about 23%, at times about 25%, at times about 27%, at times about 30%, at times about 32%, at times about 35%, at times about 38%, at times about 40%, at times about 43%, at times about 45%, at times about 47%, at times about 50%, and at times about 53% albumin out of the total amount of proteins in the composition on dry basis.

In some embodiments, the fibrinogen-comprising composition comprises about 20%, at times about 23%, at times about 25%, at times about 27%, at times about 30%, at times about 32%, at times about 35%, at times about 38%, at times about 39%, and at times about 40% albumin out of the total amount of proteins in the composition.

In some embodiments, the albumin is human serum albumin.

While the fibrinogen-comprising composition may comprise other blood derived proteins, such as fibronectin, immunoglobulin, plasminogen, vWF, Factor VIII, Antithrombin III, and serpine proteins, those, if present, are at low amounts and hence the composition described herein is referred to as a pure fibrinogen composition.

When referring to “low amounts” of other blood derived proteins it should be understood as being less than about 10% out of the total amount of proteins in the composition, at times less than about 8%, at times less than about 6%, at times less than about 5%, at times less than about 3%, at times less than about 1%, and at times less than about 0.6% out of the total amount of proteins in the composition.

Typically, the term “pure fibrinogen” refers to a composition comprising at least about 90% w/w clottable fibrinogen out of the total clottable proteins dry matter. In other words, the fibrinogen-comprising composition comprises at least 90% fibrinogen.

In some embodiments, the clottable proteins in the fibrinogen-comprising composition comprise at least about 91% fibrinogen out of the total clottable proteins dry matter, at times at least about 92% fibrinogen, at times at least about 93% fibrinogen, at times at least about 94% fibrinogen, at times at least about 95% fibrinogen, at times at least about 96% fibrinogen, at least about 97% fibrinogen, at times at least about 98% fibrinogen, at times at least about 99% fibrinogen, at least about 99.1% fibrinogen, at times at least about 99.2% fibrinogen, at least about 99.3% fibrinogen, at times at least about 99.4% fibrinogen, at least about 99.5% fibrinogen, at times at least about 99.6% fibrinogen, at least about 99.7% fibrinogen, at times at least about 99.8% fibrinogen, at least about 99.9% fibrinogen out of the total clottable proteins dry matter. In some embodiments, the clottable proteins in the fibrinogen-comprising composition comprise about 100% fibrinogen out of the total clottable proteins dry matter.

In some embodiments, the clottable proteins in the fibrinogen-comprising composition comprise between about 91% and 100% fibrinogen out of the total clottable proteins dry matter.

In some embodiments, the clottable proteins in the composition comprise between about 90% and about 99.9% fibrinogen out of the total clottable proteins dry matter, at times between about 91% and about 99.9% fibrinogen, at times between about 92% and about 99.9% fibrinogen, at times between about 93% and about 99.9% fibrinogen, at times between about 94% and about 99.9% fibrinogen, at times between about 95% and about 99.9% fibrinogen, at times between about 96% and about 99.9% fibrinogen, at times between about 97% and about 99.9% fibrinogen, at times between about 97.5% and about 99.9% fibrinogen, at times between about 98% and about 99.9% fibrinogen, at times between about 98.5% and about 99.9% fibrinogen, at times between about 99% and about 99.9% fibrinogen out of the total clottable proteins dry matter.

In some embodiments, the clottable proteins in the composition comprise between about 99% and about 99.9% fibrinogen out of the total clottable proteins dry matter, at times between about 99.1% and about 99.9%, at times between about 99.2% and about 99.9%, at times between about 99.3% and about 99.9%, at times between about 99.4% and about 99.9% and at times between about 99.5% and about 99.9% out of the total clottable proteins dry matter.

In some embodiments, the clottable proteins in the composition comprise about 91% fibrinogen out of the total clottable proteins dry matter, at times about 92% fibrinogen, at times about 93% fibrinogen, at times about 94% fibrinogen, at times about 95% fibrinogen, at times about 96% fibrinogen, at times about 97% fibrinogen, at times about 98% fibrinogen, at times about 99% fibrinogen, at times about 99.1% fibrinogen, at times about 99.2% fibrinogen, at times about 99.3% fibrinogen, at times about 99.4% fibrinogen, about 99.5% fibrinogen, at times about 99.6% fibrinogen, at times about 99.7% fibrinogen, at times about 99.8% fibrinogen and at times about 99.9% fibrinogen out of the total clottable proteins dry matter.

Examples of fibrinogen sources include, but are not limited to, recombinant fibrinogen and purified fibrinogen from plasma. Non-limiting examples of plasma sources of fibrinogen include human, porcine, bovine, and salmon.

The amount of fibrinogen can be measured by any known method in the field. For example, the fibrinogen can be determined by one or more of Clauss kinetic clotting method, Clottable fibrinogen methods, immunological methods or electrophoresis methods. The Clauss method involves measuring the rate of clot formation of a fibrinogen-containing solution. The clottable fibrinogen methods involve washing a formed clot and measuring protein content. The Clauss method measures fibrinogen that is capable of clotting in seconds (for example 5-60 seconds). The clottable protein method (involving fully clotting the fibrinogen followed by washing and then measuring the remaining protein) allows 20-60 min for clot formation. At times, this type of assay has been denoted as gravimetric fibrinogen because the clot was measured the past by weighing it. In current methods, the clotted protein is usually dissolved and measured by spectroscopy or various other protein determination methods. Determination of clot formation end point can be done by multiple ways, including, for example, clot detection by eye (this has been called the tilt-tube method when performed with plasma), viscosity-based methods (the Stago analyzer is an example) and optical methods that identify clot formation based on change in absorbance (like most hospital coagulation analyzers). Details on such methods can be found, for example, in British Journal of Haematology, 2003, 121, 396-404, the contents of which are incorporated by reference.

As described herein, it was surprisingly found that the compositions of the present disclosure are highly effective, for example, as demonstrated in the peel test, despite of the fact that they comprise a low amount of fibronectin or at times are even essentially free from fibronectin. As noted above, this is surprising specifically in view of the prior publications (e.g., Bar, et al.).

The amount of fibronectin can be measured by any method known in the field. For example, the fibronectin can be determined by an electrophoretic method, nephelometric method or immunological method. Specifically, such method may include Enzyme-Linked Immunosorbent Assay (ELISA), radial immunodiffusion, Ouchterlony immunodiffusion, and nephelometric techniques. It is of note that nephelometry method involves using antibodies (sometimes coated on particles) that aggregate and scatter light in the presence of the protein of interest.

In some embodiments, the fibrinogen-comprising composition comprises less than about 0.5% fibronectin, at times less than about 0.4%, at times less than about 0.3%, at times less than about 0.2%, at times less than about 0.1%, at times less than about 0.05% fibronectin, at times less than about 0.04%, at times less than about 0.02%, at times even less than about 0.01, at times less than about 0.005% fibronectin, at times less than about 0.004%, at times less than about 0.002%, and at times even less than about 0.001% fibronectin out of the total proteins dry matter.

In some embodiments, the fibrinogen-comprising composition comprises between about 0.001% and about 0.5% fibronectin out of the total proteins dry matter.

In some embodiments, the fibrinogen-comprising composition comprises between about 0.004% and about 0.5% fibronectin out of the total proteins dry matter.

In some embodiments, the fibrinogen-comprising composition comprises between about 0.01% and about 0.4% fibronectin, at times between about 0.01% and about 0.3%, and at times between about 0.01% and about 0.2% fibronectin out of the total proteins dry matter.

In some embodiments, the composition comprises about 0.01% fibronectin, at times about 0.05%, at times about 0.1%, at times about 0.15%, at times about 0.2%, at times about 0.25%, at times about 0.3%, at times about 0.35%, at times about 0.4% fibronectin out of the total proteins dry matter.

In some embodiments, the composition is essentially free of fibronectin.

It is of note that when referring to a fibrinogen-comprising composition free of fibronectin it encompasses a composition which comprises fibronectin in amount that is undetectable in a method used to determine fibronectin such as the methods described herein.

It is of note that recombinant fibrinogen or its composition may be at times referred to as being free of fibronectin. It is of further note that at times, recombinant fibrinogen composition may comprise fibronectin, however the amount of fibronectin is below the detection level.

In some embodiments which can be considered as aspects of the disclosure, the fibrinogen-comprising composition comprises fibrinogen and albumin at a total concentration of at least 90% (w/w) by total protein weight, wherein fibronectin is at an amount of less than about 0.5% (w/w) by total protein weight, and wherein the albumin to fibrinogen weight ratio is at least 1:15, respectively.

In some embodiments which can be considered as aspects of the disclosure, the fibrinogen-comprising composition comprises fibrinogen and albumin at a total concentration of at least about 90% (w/w) by total protein weight, wherein fibronectin is at an amount of less than about 0.5% (w/w) by total protein weight, and wherein the albumin to fibrinogen weight ratio is between 1:1 and 1:15, respectively.

In some embodiments which can be considered as aspects of the disclosure, the fibrinogen-comprising composition comprises fibrinogen and albumin at a total concentration of at least about 90% (w/w) by total protein weight, wherein fibronectin is at an amount of less than about 0.5% (w/w) by total protein weight, and wherein the albumin to fibrinogen weight ratio is between 1:1 and 1:10, respectively.

In some embodiments which can be considered as aspects of the disclosure, the fibrinogen-comprising composition comprises fibrinogen and albumin at a total concentration of at least about 90% (w/w) by total protein weight, wherein fibronectin is at an amount of less than about 0.5% (w/w) by total protein weight, and wherein the albumin to fibrinogen weight ratio is between about 1:1.6 and about 1:3, respectively.

In some embodiments which can be considered as aspects of the disclosure, the fibrinogen-comprising composition comprises fibrinogen and albumin at a total concentration of at least about 95% (w/w) by total protein weight, wherein fibronectin is at an amount of less than about 0.5% (w/w) by total protein weight, and wherein the albumin to fibrinogen weight ratio is between 1:1 and 1:10, respectively.

In some embodiments which can be considered as aspects of the disclosure, the fibrinogen-comprising composition comprises fibrinogen and albumin at a total concentration of at least about 95% (w/w) by total protein weight, wherein fibronectin is at an amount of less than about 0.5% (w/w) by total protein weight, and wherein the albumin to fibrinogen weight ratio is between about 1:1.6 and about 1:3, respectively.

In one embodiment, the fibrinogen-comprising composition comprises a cryoprecipitate comprising fibrinogen and albumin, wherein fibronectin is at an amount of less than about 0.5% by total protein weight or is absent. In one embodiment, albumin is present at a weight ratio of albumin to fibrinogen at least 1:15, respectively.

The fibrinogen-comprising composition can be in any form. In accordance with some embodiments, the fibrinogen-comprising composition is in a liquid form. When referring to a liquid form it is to be understood as one being liquid at room temperature. When in liquid form, it is in accordance with some embodiments that the liquid carrier is a buffer having an essentially neutral pH, e.g., at a range of between about 6.0 and about 8.0, at times between about 6.5 and about 8.0 and at times about at pH 7.0±0.5 and at times at pH of 7.25±0.5.

In some embodiments, the fibrinogen-comprising composition is an aqueous formulation. When referring to an “aqueous formulation” it is to be understood to encompass a blend of ingredients, in liquid or frozen, that contains water molecule.

In some embodiments, the fibrinogen-comprising composition is frozen. Prior to use, the fibrinogen-comprising composition can be thawed and thereby turn into the liquid form at room temperature.

In accordance with some embodiments, the fibrinogen-comprising composition is in a powder form or in a lyophilized form. When in powder or lyophilized form, it can be mixed with a suitable aqueous formulation such that upon use, the formulation is in the liquid form.

In some other embodiments, the fibrinogen-comprising composition is in a dry form. Typically, when referring to a composition being in a dry form it relates to a composition comprising less than 3% aqueous medium, at times less than 2% aqueous medium, and at times less than 1%. The dry form in accordance with some examples is essentially free of aqueous medium.

In some further embodiments, the composition is in a powder form.

In some embodiments, the composition is in a powder form characterized by D50 (median particle diameter, also referred to as “median particle size”) that is equal to or above 13 μm (i.e. 50% of the particles in the sample is equal to or above 13 μm) and/or D90 that is equal to or below 100 μm (i.e. 90% of the particles in the sample is equal to or below 100 μm).

The present disclosure also refers to combinations of a thrombin component with the fibrinogen-comprising composition. Such combinations with a thrombin component can be used, for example, to improve performance such as tissue adhesion.

Hence, in accordance with some aspects, the present disclosure provides a composition comprising one or more clottable proteins, albumin and thrombin, wherein the one or more clottable proteins and the albumin are at a total concentration of at least 90% by total protein weight, wherein the one or more clottable proteins comprise fibrinogen and fibronectin, with the fibronectin being at an amount of less than about 0.5% by total protein weight or being absent, wherein the albumin to the fibrinogen weight ratio is at least 1:15, respectively.

Thrombin can be obtained from various sources. In some embodiments, the thrombin is human thrombin. In some other embodiments, the thrombin is bovine thrombin. In some embodiments, the thrombin is recombinant thrombin.

The thrombin component, in accordance with some embodiments, comprises in addition to thrombin at least one of albumin, calcium, sodium chloride, glycine, acetate, or a combination thereof. In some embodiments, the thrombin component comprises about 40 mM calcium.

In some embodiments, the thrombin component comprises in addition to or in absence of thrombin at least one vitamin K-dependent coagulation factor. In some embodiments, the thrombin component comprises in addition to thrombin, an immunoglobulin, ceruloplasmin or complement C4. The vitamin K-dependent coagulation factor comprises at least one of factor II, factor VII, factor IX, factor X, protein C, protein S or combination thereof.

The composition comprising the fibrinogen-comprising composition and the thrombin is denoted herein as thrombin-comprising/containing composition and at times as “fibrin sealant” or “fibrin glue”.

In accordance with some embodiments, the fibrinogen comprising composition and thrombin are separated from each other and are combined prior to use. In accordance with such embodiments, each of the fibrinogen comprising composition and thrombin is present as a separate formulation, e.g., aqueous formulation. It is of note that each one of the fibrinogen-comprising composition and the thrombin can be frozen and thawed, after which the two are combined for use.

As described herein, the compositions in accordance with the present disclosure are hemostatic, blood clot-forming compositions. It is of note that when being in contact with a tissue and/or blood e.g., directly on a wound (e.g., a bleeding wound) or in the proximity of a wound, the compositions react to subsequently form a clot, acting as a tissue adhesive, and thereby stop bleeding.

In accordance with some embodiments, thrombin can be added to the fibrinogen-comprising composition after the lyophilization and milling of the fibrinogen-comprising composition.

In other words, the fibrinogen-comprising composition in a powder form is combined with thrombin. In some embodiments, the thrombin is milled to form a powder before being combined with a fibrinogen-comprising composition in the powder form.

In some embodiments, the powder of the thrombin composition is characterized by D50 of equal to or above 5 μm (i.e. 50% of the particles in the sample is equal or above 5 μm) and/or D90 of equal to or below 39 μm (i.e. 90% of the particles in the sample is equal to or below 39 μm).

In some embodiments, a thrombin powder is added to the fibrinogen-comprising composition powder. Hence, the power comprises thrombin and fibrinogen-comprising composition which were individually lyophilized and milled to produce a powder (herein a combined powder).

The powder can be further used, for example, as described below to be placed on an article of manufacture.

In accordance with some aspects, the present disclosure provides an article of manufacture comprising the compositions described herein.

In some embodiments, the article of manufacture comprises the fibrinogen-comprising composition. In some embodiments, a powder comprising fibrinogen comprising composition is placed on an article of manufacture.

In some embodiments, the article of manufacture comprises a composition comprising both the fibrinogen comprising composition and thrombin.

In some embodiments, the article of manufacture comprises a powder comprising the fibrinogen comprising composition and thrombin. In other words, a powder comprising fibrinogen-comprising composition and thrombin (i.e. combined powder) is placed on an article of manufacture.

In accordance with embodiments in which the article of manufacture comprises the fibrinogen-comprising composition in a dry form, thrombin can be separately applied to a wound prior to application of the article of manufacture. Thrombin may be applied by any known method in the field, including, inter alia, by spraying.

The article of manufacture as used herein is to be understood as a sterile dressing that is suitable to be placed on a tissue and specifically on a wounded tissue.

It is of note that upon application of the article of manufacture onto a wounded tissue, the dried powder is to be hydrated for a clot to be formed. In other words, the article of manufacture can be placed on a wounded tissue and/or a proximity of a wounded tissue provided that any fluid, e.g., blood, is present on the surface of the tissue to allow hydration of the powder.

In some embodiments, the article of manufacture is a wound dressing comprising the composition of the invention (with or without thrombin) and is configured to be placed at a proximity to or directly on a wound, e.g., a bleeding wound.

As used herein, the term “dressing” relates to a covering for a wound.

It is of note that an article of manufacture, specifically a wound dressing, comprising the compositions of the disclosure, refers to the compositions combined with the wound dressing.

It is of note that “combined” is to be understood as any form of association/contacting between the compositions and the dressing. This may include, without being limited thereto, any one of spraying, smearing, dipping, soaking, brushing, casting, printing, injecting, coating, or any combination thereof as further described below.

In accordance with embodiments in which the composition is in a form of a powder e.g., lyophilized, the powder can be moistened with an aqueous solution before use and combined with the wound dressing.

The term “dressing” when used herein is intended to have similar meaning as patch, or pad, all of which may interchangeably used herein.

In some embodiments, the wound dressing comprises at least one layer. In some embodiments, the wound dressing is a multilayered dressing.

In some embodiments, the wound dressing comprises at least one layer that is a backing layer.

A “backing” as used herein refers to a physical structure providing support to the composition. It is of note that the reinforcement fabric and hence the backing layer provides support/strength allowing the wound dressing to be placed on a tissue (e.g., wound) to achieve hemostasis and/or tissue adhesion.

In some embodiments, the backing layer is or comprises a reinforcement fabric.

In some embodiments, the reinforcement fabric comprises woven or knitted fabric. In some embodiments, the woven or knitted fabric is a biocompatible material. In some embodiments, the woven or knitted fabric is a bio-incompatible material.

In some embodiments, the woven or knitted fabric is an absorbable material.

In some embodiments, the wound dressing comprises a layer of an absorbable woven or knitted fabric.

In some other embodiments, the woven or knitted fabric (forming the backing layer) comprises oxidized polysaccharide(s).

In some other embodiments, the woven or knitted fabric comprises cellulose and/or derivatives thereof.

In some embodiments, the woven or knitted fabric comprises oxidized cellulose (OC), oxidized regenerated cellulose (ORC), collagen or a synthetic polymer (e.g., polyglactin 910, “PG910”).

In some embodiments, the woven or knitted fabric comprises OC.

In some other embodiments, the woven or knitted fabric comprises ORC.

The backing layer can be for example backing material known in the art, including, inter alia, those known under the tradenames INTERCEED® and SURGICEL®.

The SURGICEL® family of absorbable hemostats consists of four main product groups, with all hemostatic wound dressings commercially available from Ethicon, Inc., Somerville, N.J., a Johnson & Johnson Company. These products include SURGICEL® (the original), SURGICEL® NU-KNIT®, SURGICEL® FIBRILLAR™ and SURGICEL® SNoW™.

In some embodiments, the multilayered wound dressing comprises, in addition to the backing layer, at least one layer of an absorbable nonwoven fabric.

In some embodiments, the multilayered wound dressing comprises at least one layer of a reinforcement fabric and at least one layer of an absorbable nonwoven fabric.

The term “nonwoven fabric” as used herein typically includes, but is not limited to, bonded fabrics, formed fabrics, or engineered fabrics, e.g., manufactured by processes other than spinning, weaving or knitting.

In some embodiments, the nonwoven fabric comprises fibers comprised of aliphatic polyester polymers or copolymers of one or more monomers selected from the group consisting of lactic acid, lactide (including L-, D-, meso and D, L mixtures), glycolic acid, glycolide, e-caprolactone, p-dioxanone and trimethylene carbonate.

In some embodiments, the nonwoven fabric comprises glycolide/lactide copolymer.

In some embodiments, the multilayered dressing comprises at least one layer comprising nonwoven fabric comprising glycolide/lactide copolymer and at least one layer comprising woven or knitted fabric comprising ORC.

The absorbable nonwoven fabric may be attached to the absorbable woven or knitted fabric, either directly or indirectly. For example, the nonwoven fabric may be incorporated into the absorbable woven or knitted fabric via needle punching, calendaring, embossing or hydroentanglement, or by chemical or thermal bonding.

In some embodiments, the fibrinogen-comprising composition or the thrombin-containing composition are substantially homogeneously dispersed throughout the nonwoven fabric and/or are disposed on the surface of the nonwoven fabric.

As described herein, the article of manufacture being for example a wound dressing is placed on a tissue of a subject in need thereof. In some embodiments, the article of manufacture or the compositions is for use in topical administration.

As shown herein, it was found that application (administration) of the wound dressing as described herein is highly effective in adhesion and in initiating hemostasis.

Hence, in accordance with some aspects, the present disclosure provides a fibrinogen-comprising composition, a thrombin-containing composition (both collectively denoted herein as “composition”), an article of manufacture comprising the fibrinogen-comprising composition or an article of manufacture comprising the thrombin-containing composition (both collectively denoted herein as an article of manufacture) for use in tissue adhesive.

Tissue adherence can be measured by any method known in the art. For example, tissue adherence can be measured by peel testing. In some examples, tissue adhesiveness is considered by a value of at least 40 newtons/meter (N/m) as measured by a peel force test. In some examples, tissue adhesiveness is considered by a value of between about 40 N/m and about 90 N/m as measured by the peel force test.

In addition, and in accordance with some aspects, the present disclosure provides a fibrinogen-comprising composition, a thrombin containing composition (both collectively denoted herein as composition), an article of manufacture comprising the fibrinogen comprising composition or an article of manufacture comprising the thrombin-containing composition (both collectively denoted herein as an article of manufacture) for use in control bleeding.

In addition, and in accordance with some aspects, the present disclosure provides a fibrinogen-comprising composition, a thrombin-containing composition (both collectively denoted herein as composition), an article of manufacture comprising the fibrinogen-comprising composition or an article of manufacture comprising the thrombin-containing composition (both collectively denoted herein as an article of manufacture) for use in the treatment, inhibition, arresting or delaying bleeding or any disorder associated thereto.

The present disclosure also provides in accordance with some aspects, a method for adhering tissues, the method comprising topically administering to a subject in need thereof an effective amount of the compositions or the article of manufacture, wherein the composition is the fibrinogen-comprising composition or the thrombin-containing composition. It is of note that when referring to tissue adhesive it is for stopping bleeding and/or seal physiological leaks.

In some embodiments, the compositions and the article of manufacture are for use in sealing physiological leaks. The physiological leaks include leaks of cerebrospinal fluids (CSF), lymph, bile, gastrointestinal (GI) content, pancreatic fluid, air leak from lungs or any combination thereof.

The present disclosure also provides in accordance with some aspects, a method for controlling bleeding. Control bleeding as used herein refers to treatment, inhibition, arresting or delaying bleeding or any disorder associated thereto.

The present disclosure also provides in accordance with some aspects, a method for the treatment, inhibition, arresting or delaying bleeding or any disorder associated thereto, the method comprising topically administering to a subject in need thereof an effective amount of the composition or the article of manufacture, wherein the composition is the fibrinogen-comprising composition or the thrombin-containing composition.

The method of the invention comprises a step of topically administering to a subject in need thereof an effective amount of the compositions or the article of manufacture as described herein.

In some embodiments, the methods of the invention comprise topically administering onto a tissue of a subject in need thereof a fibrinogen-comprising composition or an article of manufacture comprising the fibrinogen-comprising composition, the composition comprising one or more clottable proteins and albumin, wherein the one or more clottable proteins and albumin are at a total concentration of at least 90% (w/w) by total protein weight, wherein the one or more clottable proteins comprise fibronectin at an amount of less than about 0.5% (w/w) by total protein weight, and wherein the one or more clottable proteins comprise fibrinogen with the albumin to fibrinogen weight ratio being at least 1:15, respectively.

In some embodiments, the methods of the invention comprise topically administering onto a tissue of a subject in need thereof a fibrinogen comprising composition or an article of manufacture comprising the fibrinogen-comprising composition, the composition comprising fibrinogen and albumin at a total concentration of at least 90% (w/w) by total protein weight, wherein fibronectin is at an amount of less than about 0.5% (w/w) by total protein weight, and wherein the albumin to fibrinogen weight ratio is at least 1:15, respectively.

In some embodiments, the methods of the invention comprise topically administering onto a tissue of a subject in need thereof, a fibrinogen-comprising composition or an article of manufacture comprising the fibrinogen-comprising composition, the composition comprising fibrinogen and albumin being at a total concentration of at least 90% (w/w) by total protein weight, wherein fibronectin is at an amount of less than about 0.5% (w/w) by total protein weight, and wherein the albumin to fibrinogen weight ratio is between 1:1 and 1:15, respectively.

In some embodiments, the methods of the invention comprise topically administering onto a tissue of a subject in need thereof a fibrinogen-comprising composition or an article of manufacture comprising the fibrinogen-comprising composition, the composition comprising fibrinogen and albumin at a total concentration of at least 90% (w/w) by total protein weight, wherein fibronectin is at an amount of less than about 0.5% (w/w) by total protein weight, and wherein the albumin to fibrinogen weight ratio is between about 1:1.6 and about 1:3, respectively.

In some embodiments, the methods of the invention comprise topically administering onto a tissue of a subject in need thereof a thrombin-containing composition or an article of manufacture comprising the thrombin-containing composition as described herein.

As used herein the term “bleeding” may refer to at least one defect in hemostasis resulting in an increased susceptibility to bleeding, e.g., hemophilia, platelet disorders, or medications affecting the hemostatic system.

In some embodiments, the bleeding is caused by an external stimulus. The bleeding may be a bleeding wound due to various reasons, e.g., personal care, trauma or during a surgical procedure.

In some embodiments, the bleeding may be at least one of injury-induced bleeding, surgery-induced bleeding or trauma-induced bleeding.

In some embodiments, the bleeding is during a surgical procedure.

In some embodiments, the methods of the invention comprise administration of the compositions or the article of manufacture during a surgical procedure.

In some embodiments, the methods of the invention may be applicable for subjects having an increased risk for bleeding.

In some embodiments, the methods of the invention may be applicable for subjects suffering from bleeding during a surgical procedure.

There are many surgical procedures in which the wound dressing of the present disclosure can be used. In some embodiments, the surgical procedure comprises abdominal surgery, cardiovascular surgery, thoracic surgery, head and neck surgery, dura surgery, spine surgery, pelvic surgery, skin and/or subcutaneous tissue procedure.

The wound dressing of the present disclosure is suitable for application (administration) on variety of wounded tissues.

In some embodiments, the wounded tissue is or comprises a bleeding artery. In some embodiments, the wounded tissue is or comprises a bleeding vein. The vein may be venae cavae.

In some embodiments, the wounded tissue is or comprises capillary bed.

In some embodiments, the methods of the invention comprise pressing/compressing the wound dressing for a time sufficient to allow adherence of the wound dressing to at least a portion of the wounded tissue.

In some embodiments, the wound dressing is for use, by compression (pressing), optionally manual compression, as an adjunct to hemostasis.

In some embodiments, the method comprises pressing (compressing) the wound dressing for a time sufficient to allow adherence of the wound dressing to at least a portion of said wounded tissue.

In some embodiments, the method comprises pressing (compressing) the wound dressing against the wounded tissue until the bleeding is essentially stopped.

In some embodiments, the method comprises pressing (compressing) the wound dressing against the wounded tissue for a time sufficient to allow hydration, tissue adherence and hemostasis.

As appreciated the time sufficient to allow any of hydration, tissue adherence and hemostasis depends on the wound to be treated and on the amount of bleeding. The time can be adjusted in order to achieve a complete hemostasis. In some embodiments, the wound dressing is compressed on at least part of a wound for about 1 minute, at times for about 3 minutes.

Further, the present disclosure provides a kit (package). In some embodiments, the kit comprises the fibrinogen-comprising composition. In some embodiments, the kit comprises the thrombin-containing composition.

In some embodiments, the kit comprises an article of manufacture.

According to some embodiments, the kit of the invention is for use in a subject suffering from bleeding or any disorder associated thereto as described herein above. It should be further appreciated that the kit of the invention may be also used for preventing bleeding in subjects having an increased risk for bleeding.

In some embodiments, the kit further comprises instructions for use of the formulation to treat, inhibit or delay bleeding or any disorder associated thereto.

The fibrinogen-comprising composition and/or the thrombin-containing composition may be stored in a liquid or frozen form and be thawed just before use.

In some embodiments, the kit is provided with instructions for use of the article of manufacture, specifically, a wound dressing in combination with the fibrinogen-comprising composition and/or thrombin-containing composition for the preparation of the wound dressing and application thereof onto the wound.

As indicated above, the method of the invention involves the administration of a therapeutically effective amount of the compositions of the invention. The “effective amount” or “therapeutically effective” for purposes disclosed herein indicate that the amount of compositions is effective to treat, inhibit or delay one or more symptoms of a disease as described herein, specifically to inhibit or stop bleeding.

As used herein, the term “subject” refers to a living organism that is treated with the formulation as described herein, including, but not limited to, any mammal, such as a human.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range disclosed and described.

It is to be understood that this invention is not limited to the particular examples, methods steps, and compositions disclosed herein as such methods, steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### NON-LIMITING EXAMPLES

### Example 1: Tissue Adherence Testing—Albumin Dose Study

The following samples shown in Table 1 were prepared by adding albumin at the indicated % to fibrinogen. The amount of fibronectin was less than about 0.4% w/w of the total protein weight. These are denoted herein as “fibrinogen-comprising composition”.

TABLE 1
Components of Tested Samples (“fibrinogen-
comprising compositions”)

| Sample # | % Albumin (w/w) | % Fibrinogen (w/w) |
|---|---|---|
| 1 | <0.5% | 99.5 |
| 2 | 23.8 | 75.8 |
| 3 | 38.5 | 61.2 |
| 4 | 55.6 | 44.2 |
| 5 | 65.2 | 34.6 |
| 6 | 100.0 | 0.0 |
| 7 | 100.0 | 0.0 |

The fibrinogen-comprising compositions with various amounts of albumin and fibrinogen were prepared at ratios as detailed in Table 1. The fibrinogen-comprising compositions were lyophilized and milled into a powder. The thrombin was lyophilized and milled separately from the fibrinogen compositions.

The fibrinogen-comprising composition powders were tested such that the amounts of clottable fibrinogen and thrombin activity were constant for all samples (but differing in the amount of albumin). The amount in Table 2 represents the amount of powder weighed out for each peel test replicate.

TABLE 2
Composition of Applied Samples

| | Amount of | | |
|---|---|---|---|
| | fibrinogen- | | |
| composition | comprising | Clottable | Amount of |
| # | compositions (mg) | Fibrinogen (mg) | Thrombin (mg) |
| 1 | 196.9 | 128 | 56 |
| 2 | 258.5 | 128 | 56 |
| 3 | 320 | 128 | 56 |
| 4 | 443.1 | 128 | 56 |
| 5 | 566.2 | 128 | 56 |
| 6 | 320 | 0 | 56 |
| 7 | 320 | 0 | 56 |

The combined powders (fibrinogen/albumin+thrombin−“thrombin-containing composition”) were tested for tissue adherence in a bovine corium peel test such that the powders were sandwiched between two pieces of corium. Specifically, corium from Lampire Biological Laboratories, Inc. (Pipersville, Pa. 18947) was mounted in a fixture/frame with approx. 4 inch×0.75 inch tissue exposed, then various amounts of powder were applied to moistened tissue mounted in the frame. A second moistened piece of corium was applied on top of the mounted tissue and a weight was applied to the backside of the tissue for 3 min at 37° C. A compression time for 3 minutes was used for all samples. After 3 min, the edge of the corium was grasped and a 90° peel test was performed across the length of the tissue. The tissue adherence results are summarized in Table 3.

TABLE 3
Results of the Tissue Adherence

| Sealant | Mean Peel | St Dev Peel |
|---|---|---|
| Composition # | Force (N/m) | Force (N/m) |
| 1 | 38.8 | 6.9 |
| 2 | 46.9 | 4.5 |
| 3 | 46.2 | 5.0 |
| 4 | 14.3 | 4.3 |
| 5 | 8.8 | 2.0 |
| 6 | 0 | 0.9 |
| 7 | 0 | 1.2 |

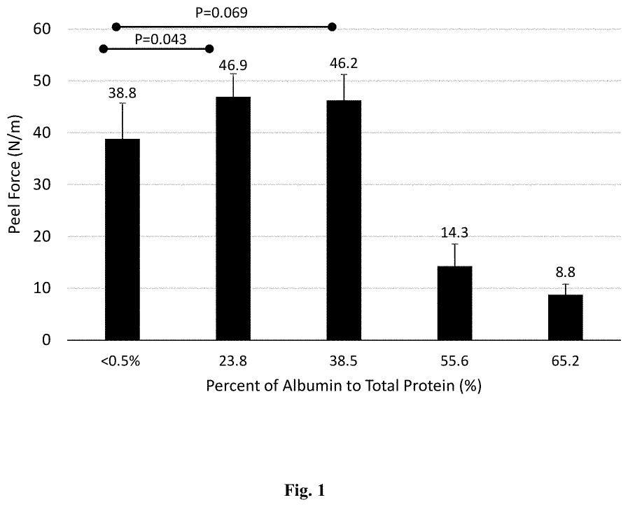

FIG. 1 shows the results of the five first compositions from Table 3 with their corresponding standard deviation values. As can be seen, the mean peel force is significantly increased in the compositions comprising 23.8% and 38.5% albumin as compared with the composition having less than 0.5% albumin. This is clearly evident from the low p-value of a t-test as shown in FIG. 1.

FIG. 1 further shows the tissue peel force as a function of % albumin to total protein in the fibrinogen-comprising compositions. It should be noted in this connection that while albumin may be present in the thrombin containing composition, its amount is relatively small and in addition, the amount of thrombin (when present) is low compared to the amount of fibrinogen so this results in a relatively small amount (marginal addition) of albumin in the final composition. In other words, while it is indicated that FIG. 1 shows the % albumin to total protein in the fibrinogen-comprising compositions, it can be also considered as the % albumin to total protein (even after addition of thrombin).

As can be seen from FIG. 1, the tissue adherence depended on the albumin % in the sample. An increase in the peel force was observed in samples comprising about 23.8% albumin and about 38.5% albumin indicting an increased tissue adherence. Increasing albumin amount to 55.6% resulted in a reduction in peel force indicative of a reduced tissue adherence.

These results surprisingly suggest that albumin affects tissue adhesion, and that addition of albumin significantly improves adhesion.

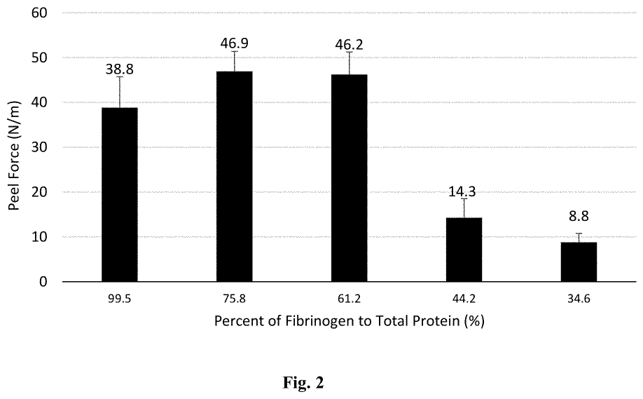

FIG. 2 shows the tissue peel force as a function of % fibrinogen to total protein. Surprisingly, the results in FIG. 2 show that in sample #2 and sample #3 (see Table 1), in which fibrinogen was lower than 99.5% or 100%, the tissue peel force was higher than the results obtained in sample #1 which include 99.5% fibrinogen.

Taken together, the results show that tissue adherence can be augmented by the addition of the albumin to the extent of 38.5%. Further dilution of fibrinogen resulted in the decrease of the tissue peel.

All the tested compositions comprised low amounts of fibronectin, that is, less than 0.5% fibronectin. The results of this example unexpectedly show that tissue adherence occurs even with low amounts of fibronectin.

### Example 2: Porcine Partial Nephrectomy Model and Tissue Adhesion Studies

This Example was designed to test hemostasis in a challenging bleeding model (see Cornum R L et al., The Journal of Urology 164, 864-867, 2000).

Powder comprising fibrinogen, albumin and thrombin was applied on a matrix containing Oxidized Regenerated Cellulose (ORC) and Polyglactin 910. The matrix characteristics also denoted herein as fibrin patch is summarized in Table 4.

TABLE 4
Coated Matrix Characteristics

| Attribute | Ranges |
|---|---|
| Water content, % | ≤3% |
| Thrombin activity, IU/cm² | 25-73 |
| Clottable proteins (fibrinogen and fibronectin) mg/cm² | 4.7-12.4 |
| Total protein, mg/cm² | 7.8-16.13 |

For the adherence test, a strip of Fibrin Patch (4 in. long×0.75 in. wide) was adhered to the moistened tissue surface, with one end of the patch attached to a load cell of an Instron tensiometer. After allowing the Fibrin Patch to adhere and to clot for 3 minutes, the patch was removed from the tissue by raising the load cell at a 90° angle from the tissue surface. The force required to separate the Fibrin Patch from the tissue substrate was recorded as force per unit width (N/m).

As shown, the coated matrix was capable of adhering to surfaces once contacted with liquid.

TABLE 5
Coated Matrixes Adhesive Strength

| | Coated matrix with fibrinogen | Coated matrix with fibrinogen |
|---|---|---|
| | and albumin less than 10% of | and albumin higher 10% of |
| Sample* | all protein solids (w/w) | all protein solids (w/w) |
| Peel test, | 79.36 (SD 7.6) | 121.19 (SD 16.57) |
| N/m | | |

*The amount of fibronectin was less than 0.4% w/w of the total protein weight.

As can be seen from Table 5 and FIG. 3 an increased adhesion was observed by a tissue adherence testing as in Example 1, in a matrix coated with a sample comprising albumin at an amount higher than 10% of all total proteins.

In addition, coated matrix showed hemostasis efficacy. Specifically, the matrix coated with formulations comprising albumin at 38.5% (corresponds to sample 3 in Table 1 above) showed a full hemostasis in a porcine partial nephrectomy model as shown in Table 6.

Technical details: (1) Mean arterial pressure (MAP) was maintained at above 60 mmHg and systolic pressures were greater than 90 mmHg during the evaluation; (2) The average kidney resection (with the capsule intact) from the caudal pole was about 3.0 cm in length and 1.0 cm in depth. The bleeding at the site was graded as severe; and (3) Complete hemostasis (4 out of 4) were achieved at a success rate of 100% at both 3 minutes and 15 minutes post-application. No rebleeding was observed.

TABLE 6
Hemostasis Outcomes in Partial Nephrectomy Model

| Test | | | |
|---|---|---|---|
| animal | Kidney | Initial Bleeding | Results with matrixes coated |
| number | side | intensity (0-4*) | with the formulation |
| 1 | right | 4 | Full hemostasis at 1^{st} attempt |
| 2 | left | 4 | Full hemostasis at 1^{st} attempt |
| 3 | right | 4 | Full hemostasis at 1^{st} attempt |
| 4 | left | 4 | Full hemostasis at 1^{st} attempt |

*“4” denotes heavy bleeding

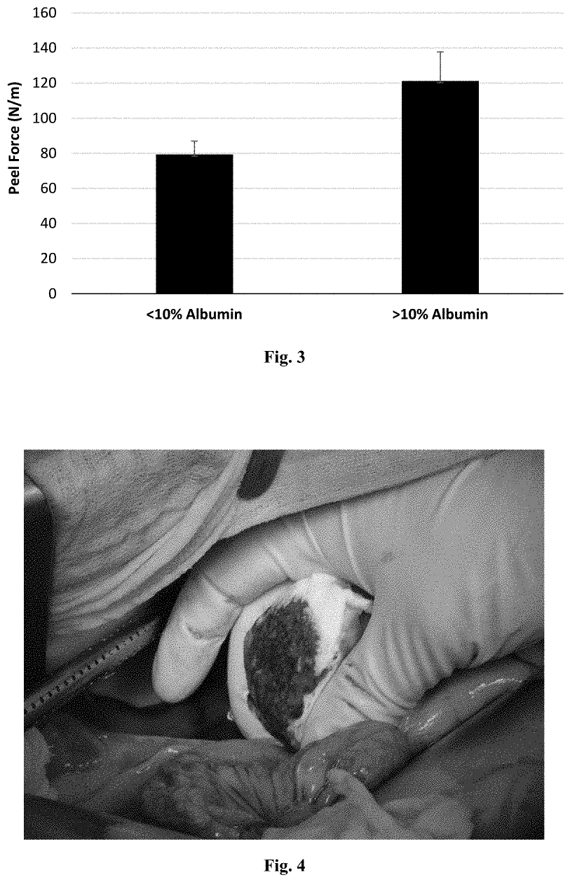

A representative image, FIG. 4, is shown. This image shows a wound dressing being a pad comprising a fibrinogen composition being applied to the bleeding surface in a porcine partial nephrectomy model. These results demonstrate that the composition was able to adhere and to stop bleeding. This shows that including albumin in the fibrinogen formulation produces a patch that is capable of adhering well to the tissue surface as well as achieving hemostasis in a partial nephrectomy model.

1. A composition comprising one or more clottable proteins and albumin, wherein the one or more clottable proteins and albumin is at a total concentration of at least 90% by total protein weight, wherein the one or more clottable proteins comprise fibrinogen and fibronectin, the fibronectin being at an amount of less than about 0.5% by total protein weight or being absent, and wherein the albumin to fibrinogen weight ratio is at least 1:15, respectively.

2. The composition of claim 1, wherein the clottable proteins comprise at least 90% fibrinogen.

3. The composition of claim 1, wherein the fibronectin is in an amount of less than about 0.3% by weight.

4. The composition of claim 1, wherein the albumin to fibrinogen weight ratio is between 1:1 and 1:15.

5. The composition of claim 1, wherein said albumin content is between about 0.5% and about 55% by weight.

6. The composition of claim 1, being in a dry form.

7. The composition of claim 1, being in a powder form.

8. The composition of claim 1, characterized by a tissue adhesiveness of at least 40 N/m as measured by a peel force test.

9. The composition of claim 1, further comprising thrombin.

10. The composition of claim 9, wherein said albumin content is between about 5% and about 43% by weight.

11. The composition of claim 10, comprising less than about 0.5% fibronectin.

12. A composition comprising one or more clottable proteins, albumin and thrombin, wherein the one or more clottable proteins and albumin are at a total concentration of at least 90% by total protein weight, wherein the one or more clottable proteins comprise fibrinogen and fibronectin, the fibronectin being at an amount of less than about 0.5% by total protein weight or being absent, wherein the albumin to fibrinogen weight ratio is at least 1:15, respectively, and wherein the composition is in a powder form.

13. An article of manufacture comprising the composition of claim 1.

14. The article of manufacture of claim 13, being a wound dressing.

15. The article of claim 13, comprising at least one layer of an absorbable woven or knitted fabric.

16. The article of manufacture of claim 13, wherein the absorbable woven or knitted fabric comprises oxidized cellulose (OC), oxidized regenerated cellulose (ORC), or combination thereof.

17. The article of manufacture of claim 15, wherein the absorbable nonwoven fabric is comprised of aliphatic polyester polymers or copolymers of one or more monomers selected from the group consisting of lactic acid, lactide (including L-, D-, meso and D, L mixtures), glycolic acid, glycolide, e-caprolactone, p-dioxanone and trimethylene carbonate.

18. A method for the treating, inhibiting, arresting or delaying bleeding or any disorder associated thereto, the method comprising topically administering onto a wounded tissue of a subject in need thereof an effective amount of the composition of claim 1.

19. The method of claim 18, wherein the bleeding is at least one of injury-induced bleeding, surgery-induced bleeding or trauma-induced bleeding

20. The method of claim 18, wherein the bleeding is during a surgical procedure.
